(19) United States
(12) Patent Application Publication
(10) Pub. No.: US 2023/0280335 A1
(43) Pub. Date: Sep. 7, 2023

(54) DROPLET ORGANOID-BASED IMMUNO-ONCOLOGY ASSAYS AND METHODS OF USING SAME

(71) Applicants: Duke University, Durham, NC (US); Xilis, Inc., Durham, NC (US)

(72) Inventors: Xiling SHEN, Durham, NC (US); Naveen NATESH, Durham, NC (US); Daniel DELUBAC, Durham, NC (US)

(21) Appl. No.: 18/114,229

(22) Filed: Feb. 25, 2023

Related U.S. Application Data
(63) Continuation of application No. PCT/US2021/060572, filed on Nov. 23, 2021.
(60) Provisional application No. 63/117,767, filed on Nov. 24, 2020.

(51) Int. Cl. G01N 33/50; A61K 35/17; G01N 1/30; C12N 5/0783; B01L 3/00

(52) U.S. Cl. CPC G01N 33/505; A61K 35/17; G01N 1/30; C12N 5/0636; B01L 3/502715; G01N 33/5017; G01N 2001/305

(57) ABSTRACT

The present disclosure describes, in part, a Micro-organosphere immune-oncology assay and methods of making and using same. The assay quickly measures the potency of effector immune cells, such as tumor infiltrating lymphocytes, at killing a patient's tumor cells. Understanding the potency of effector immune cells is critical for adoptive T cell therapy.

### CROSS REFERENCE TO RELATED APPLICATIONS

This application claims priority to U.S. Provisional Patent Application No. 63/117,767 filed on Nov. 24, 2020 in the name of Xiling Shen et al. and entitled “DROPLET ORGANOID-BASED IMMUNO-ONCOLOGY ASSAYS AND METHODS OF USING SAME,” which is hereby incorporated by reference herein in its entirety.

### BACKGROUND

Cancer therapy has been progressively moving away from the indiscriminate nature of chemotherapy and radiotherapy and towards a more targeted, patient-specific approach. This is to maximize responses in cancer patients to avoid unnecessary toxicities and holistically treat cancer, rather than iteratively treat new occurrences. In particular, immunotherapies using immune checkpoint inhibitors (ICIs), engineered T cells harboring chimeric receptors specific to particular tumor antigens (CAR T), or antibodies to inhibit immune regulatory processes have all come into the forefront of therapy. Of interest is the use of patient-derived, and possibly ex vivo engineered, T cells to specifically kill tumor cells after re-infusion. This is a particularly appealing therapy as it uses patient-derived T cells to minimize toxicity, maximize specificity, and can theoretically ablate a tumor. However, there is a clear unmet clinical need for batch-lot testing of T cells engineered against patient tumor cells. While there have been efforts for using bulk organoid systems with matched T cells as predictive of patient response, the physical barrier that bulk Matrigel imposes upon T cell infiltration of tumor cells precludes their use in diagnostics. Further, high-throughput imaging for bulk Matrigel systems have been shrouded with focal plane issues which confound fluorescence measurements and even observations of tumor-immune interaction/killing.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the development by the inventors of a technology termed (Droplet organoid-based Immuno-oncology Assay; DOIOA) which leverages droplet microfluidics for generating patient-derived tumor organoids. This assay is able to quickly test whether tumor infiltrating lymphocytes (TILs) are likely to kill patient tumors. This knowledge is critical for adoptive T cell therapy, wherein TILs from tumors are expanded and injected back into patients. The manufactured TIL potency, i.e., ability to kill cells must be validated prior to injection back into patients, and no known validation exists at this time.

Accordingly, one aspect of the present disclosure provides a method for identifying tumor cell killing by immune cells, the method comprising, consisting of, or consisting essentially of: (a) co-culturing droplet organoids and effector immune cells in a suitable medium; and (b) quantifying tumor cell killing by the effector immune cells.

Another aspect of the present disclosure provides a method for determining the potency of tumor cell killing by immune cells, the method comprising, consisting of, or consisting essentially of: (a) co-culturing droplet organoids and effector immune cells in a suitable medium; and (b) quantifying tumor cell killing by the effector immune cells.

In some embodiments, the method further comprises isolating, freezing and storing the responding effector immune cells and/or tumor cells for further analysis in a high throughput and rapid manner.

In another embodiment, the effector immune cells are selected from the group consisting of CAR-T cells, tumor infiltrating lymphocytes (TILs), Peripheral Blood Mononuclear Cells (PBMCs), T cells isolated from PBMCs, T cells isolated and expanded from tumor cells, and combinations thereof.

Still another aspect of the present disclosure provides a method of treating cancer in a patient using infiltrating lymphocytes (TIL) adoptive cell therapy (ACT), said method comprising:

validating in vitro whether TILs will kill tumor cells from said patient using any method described herein; and
administering the TILs to the patient, wherein the TILs kill the patient's tumor cells.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 illustrates Patient-Derived Micro-Organospheres formed as described herein to include dissociated primary tissue cells.

FIG. 2 is an image showing Jurkat cells adhering to and putatively killing colorectal cancer organoid cells within droplets (black dashed line) in accordance with one embodiment of the present disclosure. White arrow: Immune cell infiltrates droplet and adheres to tumor cell. Black arrow: Immune cell infiltrates droplet and settles within droplet.

FIG. 3 illustrates a generalized method of forming Patient-Derived Micro-Organospheres from primary tissue (e.g., biopsy) samples, as described herein.

FIG. 4 is an image showing a Droplet Micro-Organosphere (DMOS) generator used in the method according to one embodiment of the present disclosure.

FIG. 5 illustrates PBMC stained with Cytolight Rapid Red cytoplasmic dye and cultured with lung cancer Micro-Organospheres. Over 72 hours, there is significantly more infiltration of Matrigel by PBMCs using PMOS than bulk domes.

FIG. 6 illustrates the ability to image apoptosis/cell death within droplets in real-time using intracellular dyes.

FIG. 7 is a graph showing anti-HER2 CAR-T-induced apoptosis of cognate HER2+ colorectal cancer (CRC) droplet organoid cells in accordance with one embodiment of the present disclosure.

FIG. 8 is a graph showing TIL-induced apoptosis of matched lung tumor droplet organoid cells in accordance with one embodiment of the present disclosure.

FIG. 9 is a graph showing CAR T-specific killing of cognate HER2+ CRC organoids through decrease in reporter mCherry expression over 48-hour period in accordance with one embodiment of the present disclosure.

FIG. 10 illustrates a MOSAIC assay baseline apoptosis assessment of lung tumor Micro-Organospheres as a result of media conditions.

FIG. 11 illustrates a MOSAIC assay illustrating cell death of lung tumor Micro-Organospheres upon introduction of matched TIL as a result of droplet infiltration.

FIG. 12A illustrates the treatment of lung tumor PMOS with anti-PD1 Nivolumab with the addition of matched TILs, suggesting that Nivolumab sills lung tumor PMOS when TILs were added.

FIG. 12B illustrates the inclusion of MHC I/II blocking antibodies to assess the antigen-specific killing enhanced by Nivolumab treatment. It can be seen that when PMOS are treated with MHC block, the tumor killing effect observed in FIG. 12A disappears.

FIG. 13 illustrates that when co-cultured with tumor PMOS derived from the same patient, TIL expanded with TransAct T cell activator reagent are more cytotoxic against tumor PMOS than those expanded in the presence of irradiated PBMC.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used herein to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “biomarker” refers to a naturally occurring biological molecule present in a subject at varying concentrations useful in predicting the risk or incidence of a disease or a condition. For example, the biomarker can be a protein present in higher or lower amounts in a subject at risk for metastatic pancreatic cancer. The biomarker can include nucleic acids, ribonucleic acids, or a polypeptide used as an indicator or marker for metastatic pancreatic cancer in the subject. In some embodiments, the biomarker is a protein. A biomarker may also comprise any naturally or nonnaturally occurring polymorphism (e.g., single-nucleotide polymorphism [SNP]) present in a subject that is useful in predicting the risk or incidence of developing a disease or condition.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, peripheral blood mononuclear cells (PBMCs), mucus and tears. In one embodiment, the biological sample comprises PBMCs. A biological sample may be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It may be caused by an external factor, such as an infectious disease (e.g., viral infection), or by internal dysfunctions, such as cancer, cancer metastasis, and the like.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer comprises pancreatic cancer.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient).

As used herein, the term “effector immune cells” refer to those immune cells that defend the body of a subject during an immune response. For example, effector immune cells may include, but are not limited to, B cells and T cells (e.g., T helper cells, cytotoxic T cells), chimeric antigen receptor T cells (CAR-T cells), natural killer cells, and the like. Accordingly, in some embodiments, the effector immune cells are selected from the group consisting of CAR-T cells, tumor infiltrating lymphocytes (TILs), Peripheral Blood Mononuclear Cells (PBMCs), T cells isolated from PBMCs, T cells isolated and expanded from tumor cells, and combinations thereof.

As used herein, “potency” refers to the ability of the effector immune cells to kill tumor cells.

As defined herein, “matched” means from the same patient or autologous.

Unless otherwise defined, all technical terms used herein have the same meaning as commonly understood by one of ordinary skill in the art to which this disclosure belongs.

An important consideration for the approval and use of therapeutics is an assessment of potency, which to date has been quite difficult to delineate for cellular therapy products. According to an FDA Guidance document, potency assays shall consist of either in vitro or in vivo tests, or both, which have been specifically designed for each product so as to indicate its potency and have the following attributes: (1) indicate biological activity specific/relevant to the product, (2) measure activity of all components deemed necessary for activity, (3) provide quantitative readout, (4) results available for lot release, and (5) meet predefined acceptance and/or rejection criteria. The methods provided herein can be used to assess the potency of TIL products manufacture for each individual patient as required by the FDA. As such, according to some embodiments of the present disclosure, a portion of the tumor procured for TIL manufacture is disaggregated, and tumor cells frozen in viable fashion. When the final TIL product is available for testing, the tumor cells can be thawed, aliquoted into microdroplets and co-cultured with the TILs, and tumor cell killing quantified in high throughput and rapid fashion. The more potent the TIL products, the greater percentage of tumor cells killed.

Broadly, the present disclosure provides a method for identifying tumor cell killing by effector immune cells, the method comprising, consisting of, or consisting essentially of: (a) co-culturing Patient-Derived Micro-Organospheres (PMOSs) and effector immune cells in a suitable medium; and (b) quantifying tumor cell killing by the effector immune cells. The present disclosure further provides a method for determining the potency of tumor cell killing by effector immune cells, the method comprising, consisting of, or consisting essentially of: (a) co-culturing Patient-Derived Micro-Organospheres (PMOSs) and effector immune cells in a suitable medium; and (b) quantifying tumor cell killing by the effector immune cells. If it is concluded that the patient's effector immune cells are killing the patient's tumor cells (in the PMOSs), and hence have an acceptable potency, then treatment of the cancer patient with their effector immune cells, e.g., matched TILs, can proceed. It should be appreciated by the person skilled in the art that a conclusion that effector immune cells are killing tumor cells can vary from patient to patient and from cancer to cancer, and can correspond to at least about 10% tumor cell death, at least about 20% tumor cell death, at least about 30% tumor cell death, at least about 40% tumor cell death, at least about 50% tumor cell death, at least about 60% tumor cell death, at least about 70% tumor cell death, at least about 80% tumor cell death, at least about 90% tumor cell death, and at least about 99% tumor cell death, which can be determined using the methods and assays described herein.

By way of non-limiting examples, a suitable media or a “suitable medium” includes tumor organoid culture media. For example, the tumor organoid culture media can include basal media supplemented with growth factors such as those shown in Table I.

TABLE 1
Suitable media for the co-cultures.

| Cell Type | Illustrative Growth Factors |
|---|---|
| Colorectal cancer | A83-01, B27, EGF, [Leu15]-Gastrin I, N-Acetylcysteine, Nicotinamide, Noggin, |
| | Primocin, Prostaglandin E2, R-Spondin 1, SB202190, Y-27632 |
| Small intestine and | A83-01, B27, EGF, [Leu15]-Gastrin I, N-Acetylcysteine, N2, Nicotinamide, |
| colon | Noggin, R-Spondin 1, SB202190, Mouse Recombinant Wnt-3A, Y-27632 |
| Lung and trachea | A83-01, B27, FGF7, FGF10, N-Acetylcysteine, Nicotinamide, Noggin, R- |
| | Spondin 1, Primocin, SB202190, Y-27632 |
| Breast cancer | A83-01, B27, EGF, FGF7, FGF10, N-Acetylcysteine, Neuregulin I, |
| | Nicotinamide, Noggin, Primocin, R-Spondin 3, SB202190, Y-27632 |
| Esophageal | B27 w/o vitamin A, CultureOne supplement, EGF, FGF10, HGF, N2, Noggin |
| Liver and spleen | A83-01, B27 (w/o vitamin A), CHIR99021, EGF, FGF7, FGF10, HGF, N2, N- |
| | Acetylcysteine, Nicotinamide, R-Spondin 1, [Leu15]-Gastrin I, TGFa, Y-27632 |
| Kidney | A83-01, B27, EGF, FGF10, N-Acetylcysteine, Primocin, R-Spondin 1, Y-27632 |
| Stomach | A83-01, B27 w/o vitamin A, EGF, FGF10, [Leu15]-Gastrin I, N-Acetylcysteine, |
| | Noggin, Primocin, R-Spondin 1, Mouse Recombinant Wnt-3A, Y-27632 |
| Brainstem and | Neurobasal, 2-mercaptoethanol, B27 w/o vitamin A, Insulin, MEM-NEAA, N2 |
| cerebral | |
| Cardiac | Activin A, B27, BMP-4, CHIR99021, EGF, FGF-2, L-ascorbic acid 2-phosphate |
| | sesquimagnesium salt hydrate |
| Testicular | EGF, Insulin-Transferrin-Selenium |
| Olfactory | B27, EGF, FGF, human, Jagged-1, N2, N-Acetylcysteine, Noggin, R-Spondin 1, |
| | Mouse Recombinant Wnt-3A, Y-27632 |
| Pancreas | A83-01, B27, EGF, FGF10, [Leu15]-Gastrin I, N-Acetylcysteine, Nicotinamide, |
| | Noggin, Primocin, R-Spondin 1, Mouse Recombinant Wnt-3A |
| Sarcoma | L-Glutamine, Penicillin/Streptomycin, Fetal Bovine Serum, HI |
| Cholangiocarcinoma, | A83-01, B27, EGF, Forskolin, [Leu15]-Gastrin I, N2, N-Acetylcysteine, |
| biliary duct | Nicotinamide, R-Spondin 1, Y-27632 |
| Ovarian | 17-B Estradiol, A83-01, B27 minus Vitamin A, EGF, HGF, IGF1, N2 |
| | Supplement, N-Acetylcysteine, Neuregulin I, Nicotinamide, Noggin, R-spondin |
| | 1, SB203580 (p38i), Y-27632 |
| Liver hepatocellular | A83-01, B27, EGF, FGF10, forskolin, [Leu15]-Gastrin I, HGF, N2, N- |
| carcinoma | Acetylcysteine, Nicotinamide, R-Spondin 1, Mouse Recombinant Wnt-3A |
| Head and neck | A83-01, B27, CHIR99021, EGF, FGF2, FGF10, forskolin, N-Acetylcysteine, |
| cancer | Nicotinamide, Noggin, Prostaglandin E2, R-Spondin 1, Y-27632 |
| Liver | Non-Essential Amino Acids, Normacin, A38-01, B27, N2, N-Acetylcysteine, |
| | Nicotinamide, Y-27632, CHIR99021, EGF, HGF, TNFa, Dexamethasone |
| | (DEX) |

The present disclosure is based, in part, on a technology developed by the inventors termed (Droplet organoid-based Immuno-oncology Assay; DOIOA) that leverages droplet microfluidics for generating patient-derived tumor organoids that allows for the quick determination of whether effector immune cells, such as TILs, kill patient tumors.

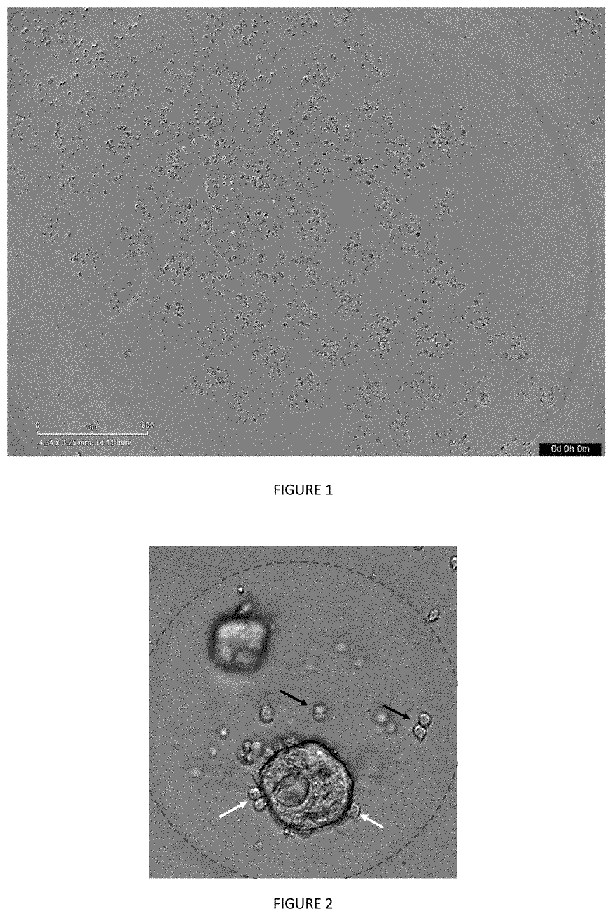

Droplet organoids, also referred to herein as Patient-Derived Micro-Organospheres (PMOSs), can be made according to PCT application no. PCT/US2020/026275, filed Apr. 1, 2020 and entitled “Methods and Apparatuses for Patient-Derived Micro-Organospheres,” the contents of which are hereby incorporated by reference in its entirety. These PMOSs may be formed from primary cells that are normal (e.g., normal organ tissue) or from tumor tissue. For example, in some variations, these methods and apparatuses may form PMOSs from cancerous tumor biopsy tissue, enabling tailored treatments that can be selected using the particular tumor tissue examined. Surprisingly, these methods and apparatuses permit the formation of hundreds, thousands or even tens of thousands (e.g., 500, 750, 1000, 2000, 5000, 10,000 or more) of PMOSs from a single tissue biopsy, within a few hours of the biopsy being removed from the patient. Dissociated primary cells from the patient biopsy may be combined with a fluid matrix material, such as a substrate basement membrane matrix (e.g., MATRIGEL), to form micro-organospheres. The resulting plurality of PMOSs may have a predefined range of sizes (such as diameters between 10 μm and 700 μm and any sub-range therewithin), and initial number of primary cells (e.g., between 1 and 1000, and in particular lower numbers of cells, such as between 1-200) (see, e.g., FIG. 1). The number of cells and/or the diameter may be controlled within, e.g., +/−5%, 10%, 15%, 20%, 25%, 30%, etc. These PMOSs, when formed as described herein, have an exceptionally high survival rate (>75%, >80%, >85%, >90%, >95%) and are stable for use and testing within a very short period of time, including within the first 1-10 days after being formed (e.g., within 1 day, within 2 days, within 3 days, within 4 days, within 5 days, within 6 days, within 7 days, within 8 days, within 9 days, within 10 days, etc.). This allows for rapid tests on a potentially huge number of patient-specific and biologically relevant PMOSs which may save critical time in developing and deploying a patient therapy, such as a cancer treatment plan.

The PMOSs described herein rapidly form three-dimensional (3D) cellular structures that replicate and correspond to the tissue environment from which they were biopsied, such as a 3D tumor microenvironment. The PMOSs described herein may also be referred to as “droplets.” Each PMOSs may further include, e.g., as part of the fluid matrix material, growth factors and structural proteins (e.g., collagen, laminin, nidogen, etc.) that may mimic the original tissue (e.g., tumor) environment. Any primary cell tissue may be used, including any tumor tissue. For example, to date, all tumor types and sites tested have successfully produced PMOSs (e.g., current success rate of 100%, n=32, including cancer of the colon, esophagus, skin (melanoma), uterus, bone (sarcoma), kidney, ovary, lung, and breast from the primary site or metastatic sites including liver, omentum, and diaphragm). The tissue types used to successfully generate Micro-Organopheres may be metastasized from other locations. In some variations the PMOSs described herein can be grown from fine needle aspirate (FNA) or from circulating tumor cells (CTCs), e.g., from a liquid biopsy. Proliferation and growth is typically seen in as few as 3-4 days, and the PMOSs can be maintained and passaged for months, or they may be cryopreserved and/or used for assays immediately (e.g., within the first 7-10 days).

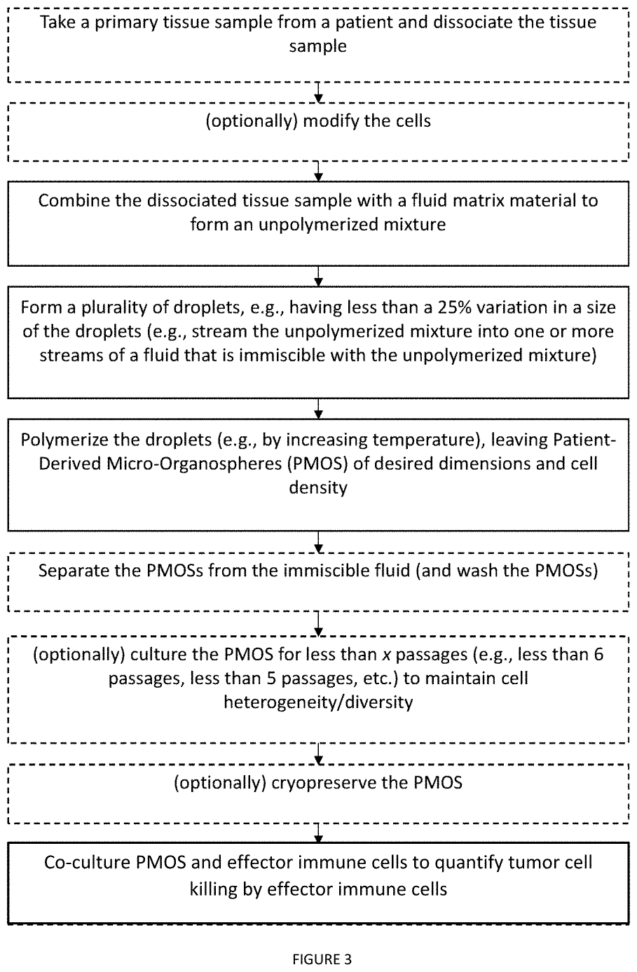

Further described herein are methods of forming the PMOSs. Reference is to the generalized schematic shown in FIG. 3, wherein the dashed boxes are optional. Generally, these methods include combining dissociated primary tissue cells including, but not limited to, cancer/abnormal tissue calls and normal tissue cells, with a liquid matrix material to form an unpolymerized material, and then polymerizing the unpolymerized material to form micro-Organospheres that are typically less than about 1000 μm (e.g., less than about 900 μm, less than about 800 μm, less than about 700 μm, less than about 600 μm, and in particular, less than about 500 μm) in diameter in which the dissociated primary tissue cells are distributed. The number of dissociated cells per micro-Organosphere may be within a predetermined range, as mentioned above (e.g., between about 1 and about 500 cells, between about 1-200 cells, between about 1-150 cells, between about 100 cells, between about 1-75 cells, between about 1-50 cells, between about 1-30 cells, between about 1-20 cells, between about 1-10 cells, between about 5-15 cells, between about 20-30 cells, between about 30-50 cells, between about 40-60 cells, between about 50-70 cell, between about 60-80 cells, between about 70-90 cells, between about 80-100 cells, between about 90-110 cells, etc., including about 1 cell, about 10 cells, about 20 cells, about 30 cells, about 40 cells, about 50 cells, about 60 cells, about 70 cells, etc.). Any of these methods may be configured as described herein to produce Micro-Organospheres of repeatable size for example, having a narrow distribution of sizes.

The dissociated cells may be freshly biopsied and may be dissociated in any appropriate manner, including mechanical and/or chemical dissociation (e.g., enzymatic disaggregation by using one or more enzymes, such as collagenase, trypsin, etc.). The dissociated cells may optionally be treated, selected and/or modified. For example, the cells may be sorted or selected to identify and/or isolate cells having one or more characteristics (e.g., size, morphology, etc.). The cells may be marked (e.g., with one or more markers) that may be used to aid in selection. In some variations the cells may be sorted by a known cell-sorting technology, including but not limited to microfluidic cell sorting, fluorescent activated cell sorting, magnetic activated cell sorting, etc. Alternatively, the cells may be used without sorting.

In some variations, the dissociated cells may be modified by treatment with one or more agents. For example, the cells may be genetically modified. In some variations the cells may be modified using CRISPR-Cas9 or other genetic editing techniques. In some variations the cells may be transfected by any appropriate method (e.g., electroporation, cell squeezing, nanoparticle injection, magnetofection, chemical transfection, viral transfection, etc.), including transfection with plasmids, RNA, siRNA, etc. Alternatively, the cells may be used without modification.

The unpolymerized mixture can comprise, consist of, or consist essentially of the dissociated cells and fluid (e.g., liquid) matrix material. The unpolymerized mixture can further include at least one additional material. For example, the at least one additional material may include additional cell or tissue types, including support cells. The additional cells or tissue may originate from a different biopsy (e.g., primary cells from a different dissociated tissue) and/or cultured cells. The additional cells may be, for example immune cells, stromal cells, endothelial cells, etc. The at least one additional material may include medium (e.g., growth medium, freezing medium, etc.), growth factors, support network molecules (e.g., collagen, glycoproteins, extracellular matrix, etc.), or the like. In some variations the at least one additional material may include a drug composition. In some variations the unpolymerized mixture consists of only the dissociated tissue sample (e.g., primary cells) and the fluid matrix material. The methods may rapidly form a plurality of Patient-Derived Micro-Organospheres from a single tissue biopsy, so that greater than about 500 Patient-Derived Micro-Organospheres are formed per biopsy (e.g., greater than about 600, greater than about 700, greater than about 800, greater than about 900, greater than about 1000, greater than about 2000, greater than about 2500, greater than about 3000, greater than about 4000, greater than about 5000, greater than about 6000, greater than about 7000, greater than about 8000, greater than about 9000, greater than about 10,000, greater than about 11,000, greater than about 12,000, etc.). The biopsy may be a standard size biopsy, such as an 18 G (e.g., 14 G, 16 G, 18 G, etc.) core biopsy. For example, the volume of tissue removed by biopsy and used to form the plurality of Patient-Derived Micro-Organospheres may be a small cylinder (taken with a biopsy needle) of between about 1/32 and ⅛ of an inch diameter and about ¾ inch to ¼ inch long, such as a cylinder of about 1/16 inch diameter by ½ inch long. The biopsy may be taken by needle biopsy, e.g., by core needle biopsy. In some variations the biopsy may be taken by fine needle aspiration. Other biopsy types that may be used include shave biopsy, punch biopsy, incisional biopsy, excisional biopsy, and the like. Typically, the material from a single patient biopsy may be used to generate the plurality (e.g., greater than about 2000, greater than about 5000, greater than about 7500, greater than about 10,000, etc.) of Patient-Derived Micro-Organospheres as described herein.

The plurality of Patient-Derived Micro-Organospheres may be formed using an apparatus (as described herein) that may be configured to generate this large number of highly regular (size, cell number, etc.) Micro-Organospheres as described herein. In some variations these methods and apparatuses may generate the plurality of Micro-Organospheres at a rapid rate (e.g., greater than about 1 Micro-Organosphere per minute, greater than about 1 Micro-Organosphere per 10 seconds, greater than about 1 Micro-Organosphere per 5 seconds, greater than about 1 Micro-Organosphere per 2 seconds, greater than about 1 Micro-Organosphere per second, greater than about 2 Micro-Organospheres per second, greater than about 3 Micro-Organospheres per second, greater than about 4 Micro-Organospheres per second, greater than about 5 Micro-Organospheres per second, greater than about 10 Micro-Organospheres per second, greater than 50 Micro-Organospheres per second, greater than 100 Micro-Organospheres per second, greater than 125 Micro-Organospheres per second, etc.). In some variations, these methods may be performed by combining the unpolymerized mixture with an additional material (e.g., liquid material) that is immiscible with the unpolymerized material. The method and apparatus may control the size and/or cell density of the Micro-Organospheres by, at least in part, controlling the flow of one or more of the unpolymerized mixture (i.e., the dissociated tissue and fluid matrix) and an additional material that is immiscible, such as a hydrophobic material, oil, etc., with the unpolymerized mixture. For example, in some variations, these methods may be performed using a microfluidics apparatus. In some variations, multiple Micro-Organospheres may be formed in parallel (e.g., 2 in parallel, 3 in parallel, 4 in parallel, etc.). The same apparatus may therefore include multiple parallel channels, which may be coupled to the same source of unpolymerized material, or the same source of dissociated primary tissue and/or source of fluid matrix. The unpolymerized material may be polymerized in order to form the Patient-Derived Micro-Organospheres in a variety of different ways. In some variations the methods may include polymerizing the Micro-Organospheres by changing the temperature (e.g., raising the temperature above a threshold value, such as, for example greater than about 20° C., greater than about 25° C., greater than about 30° C., greater than about 35° C., etc.). It should be appreciated by the person skilled in the art that other apparatuses configured to generate Micro-Organospheres can alternatively be used.

Once polymerized, the Patient-Derived Micro-Organospheres may be allowed to grow, e.g., by culturing, and/or may be assayed either before or after culturing, and/or may be cryopreserved either before or after culturing. The Patient-Derived Micro Organospheres may be cultured for any appropriate length of time, but in particular may be cultured for between 1 day and 10 days (e.g., between 1 day and 9 days, between 1 day and 8 days, between 1 day and 7 days, between 1 day and 6 days, between 3 days and 9 days, between 3 days and 8 days, between 3 days and 7 days, etc.). In some variations, the Patient-Derived Micro-Organospheres may be cryopreserved or assayed before six passages, which may preserve the heterogeneity of the cells within the Patient-Derived Micro-Organospheres; limiting the number of passages may prevent the faster-dividing cells from outpacing more slowly dividing cells (see, e.g., FIG. 2).

In general, since the same patient biopsy may provide a high number of cells (e.g., greater than 2,000, greater than 3,000, greater than 4,000, greater than 5,000, greater than 6,000, greater than 7,000, greater than 8,000, greater than 9,000, greater than 10,000, etc.), some portion of the Patient-Derived Micro-Organospheres may be cryopreserved (e.g., at least 50%) while some are cultured and/or assayed. As will be described in greater detail herein, cryopreserved Patient-Derived Micro-Organospheres may be banked and used (e.g., assayed, passaged, etc.) later.

Accordingly, described herein are methods of forming a plurality of Patient-Derived Micro-Organospheres. For example, a method of forming a plurality of Patient-Derived Micro-Organospheres may include: combining a dissociated tissue sample and a fluid matrix material to form an unpolymerized mixture; forming a plurality of droplets of the unpolymerized mixture; and polymerizing the droplets to form a plurality of Patient-Derived Micro-Organospheres each having a diameter of between 50 and 500 μm with between 1 and 200 dissociated cells distributed therein.

An embodiment of the method of forming a plurality of Patient-Derived Micro-Organospheres may include: combining a dissociated tissue sample and a fluid matrix material to form an unpolymerized mixture; forming a plurality of droplets from a continuous stream of the unpolymerized mixture wherein the droplets have less than a 25% variation in size; and polymerizing the droplets by warming to form a plurality of Patient-Derived Micro-Organospheres each having between 1 and 200 dissociated cells distributed within each Patient-Derived Micro-Organosphere. In another embodiment, the method for forming a plurality of Patient-Derived Micro-Organospheres may include: combining a dissociated tissue sample and a fluid matrix material to form an unpolymerized mixture; forming a plurality of droplets having less than a 25% variation in size of the droplets by converging a stream of the unpolymerized mixture with one or more streams of a fluid that is immiscible with the unpolymerized mixture; polymerizing the droplets to form a plurality of Patient-Derived Micro-Organospheres having a diameter of between 50 and 500 μm with between 1 and 200 dissociated cells distributed therein; and separating the plurality of Patient-Derived Micro-Organospheres from the fluid that is immiscible. Any of these methods may include modifying the cells within the dissociated tissue sample prior to forming the droplets.

Forming the plurality of droplets may comprise forming a plurality of droplets of the unpolymerized mixture of uniform size with less than about 25% variation in size (e.g., less than about 20% variation in size, less than about 15% variation in size, less than about 10% variation in size, less than about 8% variation in size, less than about 5% variation in size, etc.). The variations in size may also be described as a narrow distribution of size variation. For example, the distribution of sizes may include a Patient-Derived Micro-Organospheres size distribution (e.g., Micro-Organosphere diameter vs. the number of formed Micro-Organospheres) having a low standard deviation (e.g., a standard deviation of 15% or less, a standard deviation of 12% or less, a standard deviation of 10% or less, a standard deviation of 8% or less, a standard deviation of 6% or less, a standard deviation of 5% or less, etc.).

Any of these methods may also include plating or distributing the Patient-Derived Micro-Organospheres. For example, in some variations, the method may include combining Patient-Derived Micro-Organospheres from various sources into a receptacle prior to assaying. For example, the Micro-Organospheres may be placed into a multi-well plate. Thus, any of these methods may include dispensing the Patient-Derived Micro-Organospheres into a multi-well plate prior to assaying the Patient-Derived Micro-Organospheres. One or more (or in some variations in equal amounts of) Patient-Derived Micro-Organospheres may be included per well. In some variations applying the Patient-Derived Micro-Organospheres into a receptacle may include placing the Micro-Organopsheres into a plurality of chambers that are separated by an at least partially permeable membrane to permit circulation of supernatant material between the chambers. This may allow the Patient-Derived Micro-Organospheres to share the same supernatant.

Any appropriate tissue sample may be used in any of the methods described herein. In some variations, the tissue sample comprises a biopsy sample from a metastatic tumor. For example, a tissue sample may comprise a clinical tumor sample; the clinical tumor sample may comprise both cancer cells and stroma cells. In some variations, the tissue sample comprises tumor cells and one or more of: mesenchymal cells, endothelial cells, and immune cells.

Any of the methods described herein may include initially distributing the dissociated cells from the tissue biopsy uniformly, or in some variations non-uniformly, throughout the fluid matrix material, in any appropriate concentration. For example, in some variations, the methods described herein may include combining the dissociated tissue sample and the fluid matrix material so that the dissociated tissue cells are distributed within the fluid matrix material at a density of less than 1×10⁷ cells/ml (e.g., less than 9×10⁶ cells/ml, 7×10⁶ cells/ml, 5×10⁶ cells/ml, 3×10⁶ cells/ml, 1×10⁶ cells/ml, 9×10⁵ cells/ml, 7×10⁵ cells/ml, 5×10⁵ cells/ml, etc.).

In general, forming the droplet may comprise forming the droplet from a continuous stream of the unpolymerized mixture. For example, forming the droplet may comprise applying one or more convergent streams of a fluid that is immiscible with the unpolymerized mixture to the stream of unpolymerized mixture. The streams may be combined in a microfluidic device, e.g., a device having a plurality of converging channels into which the unpolymerized mixture and the immiscible fluid interact to form droplets having a precisely controlled volume. In some variations the droplets are formed (e.g., pinched off) in an excess of the immiscible material, and the droplets may be concurrently and/or subsequently polymerized to form the Patient-Derived Micro-Organospheres. For example, the region in which the streams converge may be configured to polymerize the unpolymerized mixture after the droplet has been formed, e.g., by heating, and/or the regions downstream may be configured to polymerize the unpolymerized mixture after the droplets have been formed and are surrounded by the immiscible material. In some variations the immiscible material is heated (or alternatively cooled) to a temperature that promotes polymerization of the unpolymerized material, forming the Patient-Derived Micro-Organospheres. For example, polymerizing may comprise heating the droplet to greater than 35° C.

Thus, in any of these methods, forming the droplet may include forming the droplet in a fluid that is immiscible with the unpolymerized mixture. Further, any of these methods may include separating the immiscible fluid from the Patient-Derived Micro-Organospheres. For example, any of these methods may include removing the immiscible fluid from the Patient-Derived Micro-Organospheres. In general, an immiscible fluid may include a liquid (e.g., oil, polymer, etc.), including in particular a hydrophobic material or other material that is immiscible with the unpolymerized (e.g., aqueous) material.

The fluid matrix material may be a synthetic or non-synthetic unpolymerized basement membrane material. In some variations the unpolymerized basement material may comprise a polymeric hydrogel. In some variations the fluid matrix material may comprise a MATRIGEL. Thus, combining the dissociated tissue sample and the fluid matrix material may comprise combining the dissociated tissue sample with a basement membrane matrix.

The tissue sample may be combined with the fluid matrix material within six hours of removing the tissue sample from the patient or sooner (e.g., within about 5 hours, within about 4 hours, within about 3 hours, within about 2 hours, within about 1 hour, etc.).

Also described herein are methods of assaying or preserving Patient-Derived Micro-Organospheres. For example, an embodiment of the method described herein may include: combining a dissociated tissue sample and a fluid matrix material to form an unpolymerized mixture; forming a plurality of droplets of the unpolymerized mixture having less than a 25% variation in a size of the droplets; polymerizing the droplets to form a plurality of Patient-Derived Micro-Organospheres having a diameter of between 50 and 700 μm with between 1 and 1000 dissociated cells distributed therein; and assaying or cryopreserving the plurality of Patient-Derived Micro-Organospheres.

In some variations an embodiment of the method described herein may include: combining a dissociated tissue sample and a fluid matrix material to form an unpolymerized mixture; forming a plurality of droplets of the unpolymerized mixture; polymerizing the droplets to form a plurality of Patient-Derived Micro-Organospheres each having a diameter of between 50 and 500 μm with between 1 and 200 dissociated cells distributed therein; and cryopreserving or assaying the plurality of Patient-Derived Micro-Organospheres within 15 days, wherein the Micro-Organospheres are assayed to determine the effect of one or more agents on the cells within the Patient-Derived Micro-Organospheres.

Another embodiment of the method described herein may include: combining a dissociated tissue sample and a fluid matrix material to form an unpolymerized mixture; forming a plurality of droplets having less than a 25% variation in a size of the droplets by converging a stream of the unpolymerized mixture with one or more streams of a fluid that is immiscible with the unpolymerized mixture; polymerizing the droplets by warming to form Patient-Derived Micro-Organospheres each having a diameter of between 50 and 500 μm with between 1 and 200 dissociated cells distributed therein; and assaying or cryopreserving the Patient-Derived Micro-Organospheres before six passages, whereby heterogeneity of the cells within the Patient-Derived Micro-Organospheres is maintained, and wherein assaying comprises assaying in order to determine the effect of one or more agents on the cells within the Patient-Derived Micro-Organospheres.

In any of these methods, the plurality of Patient-Derived Micro-Organospheres may be cryopreserved or assayed before six passages, whereby heterogeneity of the cells within the Patient-Derived Micro-Organospheres is maintained. Any of these methods may further include modifying the cells within the dissociated tissue sample prior to forming the droplets. Forming the droplets may include forming a plurality of droplets of the unpolymerized mixture of uniform size with less than about 25% variation in size (e.g., less than about 20%, less than about 15%, less than about 10%, less than about 7%, less than about 5%, etc.).

In any of these methods the Patient-Derived Micro-Organospheres may be assayed. An assay may generally include exposing or treating individual Patient-Derived Micro-Organospheres to conditions (e.g., drug compositions) to determine if the drug composition has an effect on the cells of the Patient-Derived Micro-Organospheres as well as what effect the drug composition has on the Patient-Derived Micro-Organospheres. Assays may include exposing a subset of the Patient-Derived Micro-Organospheres (individually or in groups) to one or more concentrations of a drug composition. For example, in one embodiment, an assay includes allowing the Patient-Derived Micro-Organospheres to remain exposed to a drug composition for a predetermined time period (e.g., minutes, hours, days, etc.), optionally removing the drug composition, then culturing the Patient-Derived Micro-Organospheres for a predetermined time period. Thereafter, the Patient-Derived Micro-Organospheres may be examined to identify any effects, including in particular toxicity to the cells in the Patient-Derived Micro-Organospheres, or a change in morphology and/or growth of the cells in the Patient-Derived Micro-Organospheres. In some variations assaying may include marking (e.g., by immunohistochemistry) live or fixed cells within the Patient-Derived Micro-Organospheres. Cells may be assayed (e.g., examined) manually or automatically. For example, cells may be examined to determine any toxicity (cell death) using an automated reader apparatus. In some variations assaying the plurality of Patient-Derived Micro-Organospheres may include sampling one or more of a supernatant, an environment, and a microenvironment of the Patient-Derived Micro-Organosphere for secreted factors and other effects. In any of these variations, the Patient-Derived Micro-Organospheres may be recovered following the assay for further assaying, expansion or preservation (e.g., cryopreserving, fixation, etc.) for subsequent examination.

As mentioned, virtually any assay may be used. For example, genomic, transcriptomic, proteomics, or meta-genomic markers (such as methylation) may be assayed using the PMOSs described herein. Thus, any of these compositions and methods described herein may be used to identify or examine one or more markers and biological/physiological pathways, including, for example, exosomes, which may assist in identifying drugs and/or therapies for patient treatment.

Any of these methods may include culturing the Patient-Derived Micro-Organospheres for an appropriate length of time, as mentioned above (e.g., culturing the Patient-Derived Micro-Organospheres for between 2-14 days before assaying). For example, these methods may include removing the immiscible fluid from the Patient-Derived Micro-Organospheres before culturing. In some variations, culturing the Patient-Derived Micro-Organospheres comprises culturing the Patient-Derived Micro-Organospheres in suspension. In general, assaying the Patient-Derived Micro-Organospheres may comprise genomically, transcriptomically, epigenomically and/or metabolically analyzing the cells in the Patient-Derived Micro-Organospheres before and/or after assaying or cryopreserving the Patient-Derived Micro-Organospheres. Any of these methods may include assaying the Patient-Derived Micro-Organosphere by exposing the Patient-Derived Micro-Organosphere to a drug (e.g., drug composition).

In any of these methods, assaying may comprise visually assaying the effect of the one or more agents on the cells in the Patient-Derived Micro-Organosphere either manually and/or automatically. Any of these methods may include marking or labeling cells in the Patient-Derived Micro-Organospheres for visualization. For example, assaying may include fluorescently assaying the effect of the one or more agents on the cells.

The Patient-Derived Micro-Organospheres described herein are themselves novel and may be characterized as a composition of matter. For example, a composition of matter may comprise a plurality of cryopreserved Patient-Derived Micro-Organospheres, wherein each Patient-Derived Micro-Organosphere has a substantially spherical shape having a diameter of between 50 μm and 500 μm and comprises a polymerized base material, and between about 1 and 1000 dissociated primary cells distributed within the base material that have been passaged less than six times, whereby heterogeneity of the cells within the Patient-Derived Micro-Organospheres is maintained.

Also described herein are compositions of matter comprising a plurality of cryopreserved Patient-Derived Micro-Organospheres, wherein each Patient-Derived Micro-Organosphere has a substantially spherical shape having a diameter of between 50 μm and 500 μm, wherein the Patient-Derived Micro-Organospheres have less than a 25% variation in size, and wherein each Patient-Derived Micro-Organosphere comprises a polymerized base material, and between about 1 and 500 dissociated primary cells distributed within the base material that have been passaged less than six times, whereby heterogeneity of the cells within the Patient-Derived Micro-Organospheres is maintained.

The primary cells may be primary tumor cells. For example, the dissociated primary cells may have been genetically or biochemically modified. The plurality of cryopreserved Patient-Derived Micro-Organospheres may have a uniform size with less than 25% variation in size. In some variations the plurality of cryopreserved Patient-Derived Micro-Organospheres may comprise Patient-Derived Micro-Organospheres from various sources. In any of these Micro-Organospheres, the majority of cells in each Micro-Organosphere may comprise cells that are not stem cells. In some variations, the primary cells comprise metastatic tumor cells. The primary cells may comprise both cancer cells and stroma cells. In some variations, the primary cells comprise tumor cells and one or more of: mesenchymal cells, endothelial cells, and immune cells. The primary cells may be distributed within the polymerized base material at a density of less than, e.g., 5×10⁷ cells/ml, 1×10⁷ cells/ml, 9×10⁶ cells/ml, 7×10⁶ cells/ml, 5×10⁶ cells/ml, 1×10⁶ cells/ml, 9×10⁵ cells/ml, 7×10⁵ cells/ml, 5×10⁵ cells/ml, 1×10⁵ cells/ml, etc.

In general, the polymerized base material may comprise a basement membrane matrix (e.g., MATRIGEL). In some variations the polymerized base material comprises a synthetic material.

The Micro-Organospheres may have a diameter of between 50 μm and 1000 μm, or more preferably between 50 μm and 700 μm, or more preferably between 50 μm and 500 μm, or between 50 μm and 400 μm, or between 50 μm and 300 μm, or between 50 μm and 250 μm, etc. (e.g., less than about 500 μm, less than about 400 μm, less than about 300 μm, less than about 250 μm, less than about 200 μm, etc.).

As mentioned, the Patient-Derived Micro-Organospheres described herein may include any appropriate number of primary tissue cells initially in each Patient-Derived Micro-Organosphere, for example less than about 200 primary cells, or more preferably less than about 150 primary cells, or more preferably less than about 100 primary cells, or more preferably less than about 75 primary cells, or less than about 50 cells, or less than about 30 cells, or less than about 25 cells, or less than about 20 cells or less than about 10 cell, or less than about 5 cells, etc.). In some variations each Patient-Derived Micro-Organosphere includes between about 1 and 500 cells, between about 1-400 cells, between bout 1-300 cells, between about 1-200 cells, between about 1-150 cells, between about 1-100 cells between about 1-75 cells, between about 1-50 cells, between about 1-30 cells, between about 1-25 cells, between about 1-20 cells, etc.

Also described herein are apparatuses for forming Patient-Derived Micro-Organospheres, and methods of operating these apparatuses to form the Patient-Derived Micro-Organospheres. For example, described herein are methods of operating a Patient-Derived Micro-Organosphere forming apparatus comprising: receiving an unpolymerized mixture comprising a chilled mixture of a dissociated tissue sample and a first fluid matrix material in a first port; receiving a second fluid that is immiscible with the unpolymerized mixture in a second port; combining a stream of the unpolymerized mixture with one or more streams of the second fluid to form droplets of the unpolymerized mixture having a uniform size that varies by less than 25%; and polymerizing the droplets of the unpolymerized mixture to form a plurality of Patient-Derived Micro-Organospheres.

An embodiment of the method of operating a Patient-Derived Micro-Organosphere forming apparatus may include: receiving an unpolymerized mixture comprising a chilled mixture of a dissociated tissue sample and a first fluid matrix material in a first port; receiving a second fluid that is immiscible with the unpolymerized mixture in a second port; combining a stream of the unpolymerized mixture at a first rate with one or more streams of the second fluid at a second rate to form droplets of the unpolymerized mixture having a uniform size that varies by less than 25%, wherein the droplets are between 50 μm and 500 μm diameter; and polymerizing the droplets of the unpolymerized mixture to form a plurality of Patient-Derived Micro-Organospheres.

Any of these methods may include communicatively connecting a first reservoir containing the unpolymerized mixture in fluid communication with the first port. Any of these methods may further include combining the dissociated tissue sample and the first fluid matrix material to form the unpolymerized mixture. In some variations, the method includes adding the unpolymerized mixture to a first reservoir in fluid communication with the first port. These methods may include communicatively connecting a second reservoir containing the second fluid in fluid communication with the second port. Any of these methods may further include adding the second fluid to a second reservoir in fluid communication with the second port. In some variations, receiving the second fluid comprises receiving an oil. Combining the streams may comprise driving the stream of the unpolymerized mixture at a first flow rate across one or more streams of the second fluid which is traveling a second flow rate. In some variations the first flow rate is greater than the second flow rate. Either or both the flow rate and/or the amount of material (e.g., the unpolymerized mixture) may be present in smaller amounts than the second fluid, so that the unpolymerized mixture is encapsulated in a precisely controlled droplet, as described herein, that may then be polymerized, e.g., within the second fluid. In some variations, combining the streams comprises driving the stream of the unpolymerized mixture across a junction into which the one or more streams of the second fluid also converge. Polymerizing the droplets may comprise heating the droplets to greater than a temperature at which the unpolymerized material polymerizes (e.g., greater than about 25° C., greater than about 30° C., greater than about 35° C., etc.). In one embodiment, a droplet Micro-Organosphere forming assembly is used, said assembly including one or more microfluidic chips or structures that form and control the streams of the first fluid matrix material and the second fluid and forms the actual droplets.

In general, these methods may further include separating the second fluid (e.g., the immiscible fluid) from the plurality of Patient-Derived Micro-Organospheres. This fluid may be manually or automatically separated. For example, the second (immiscible) fluid may be removed by washing, filtering, or any other appropriate method.

In some embodiments, and as described herein, these methods further comprise isolating, freezing and storing the responding effector immune cells and/or tumor cells for further analysis in a high throughput and rapid manner.

In still another aspect, a Micro-Organosphere assay for immune cytotoxicity (MOSAIC) assay to measure potency is described, wherein the MOSAIC assay and method of using same can be used to quantify effector immune cell toxicity against matched tumor cell PMOSs. For example, in one embodiment, the MOSAIC assay can be used to quantify TIL cytotoxicity, including rapid-expansion phase TIL toxicity, against matched tumor cell PMOSs. By culturing tumor cell PMOSs with matched effector immune cells in the presence of fluorescent dyes, e.g., Annexin V Green, immune-induced tumor cell apoptosis can be quantified and imaged. Goals of the assay include, but are not limited to, providing a diagnostic assay which can differentiate between immunotherapy responders and non-responders, identifying and quantifying tumor killing by immune cells, and a fast and reproducible assay from patient-derived tumor droplet organoids and matched TILs.

The MOSAIC assay and method of using same comprises co-culturing tumor cell PMOSs, produced according to any method described herein, and effector immune cells in a suitable medium. In an embodiment, the tumor cell PMOSs and the effector immune cells are matched. In another embodiment, the effector immune cells comprise TILs and the tumor cell PMOSs and the TILs are matched. In still another embodiment, the effector immune cells comprise rapid expansion phase (REP) TILs, and the REP TILs and the tumor cell PMOSs are matched. Rapid expansion phase TILs can be obtained by subjecting the TILs to irradiated PBMC feeder cells or TransAct T cell activator reagent. In addition, cytokines such as IL-2 can be used to activate the T cells. The assay is effectuated in the presence of fluorescent dyes, e.g., intracellular fluorescent dyes, to quantify apoptosis and cell death by effector immune cells in real time. Fluorescent dyes include, but are not limited to, Annexin V Green, Caspase 3/7, Cytotox, Cytotox Red, Cytolight Red, orange color, or near-infrared color dyes. The use of fluorescent microscopes for real-time imaging is well known in the art. In one variation, an Incucyte device can be used for imaging in real time. For example, the co-culturing can be performed in a well plate (e.g., a 96-well plate) and fluorescent images obtained over time. The assay method can further comprise measuring baseline apoptosis as a function of the media conditions, as understood by the person skilled in the art.

Advantageously, the MOSAIC assay can be used in a combination setting wherein tumor cell PMOSs, produced according to any method described herein, are treated with at least one drug in the presence of effector immune cells in a suitable medium. In an embodiment, the tumor cell PMOSs and the effector immune cells are matched. In another embodiment, the effector immune cells comprise TILs and the tumor cell PMOSs and the TILs are matched. The assay is effectuated in the presence of fluorescent dyes to quantify apoptosis and cell death by effector immune cells in real time.

It should be appreciated by the person skilled in the art that the tumor cell PMOSs and the effector immune cells are disclosed as being matched, or autologous, in the MOSAIC assay, however it is contemplated that there may be instances where they are not matched or autologous.

Another aspect of the present disclosure provides all that is described and illustrated herein.

The following Examples are provided by way of illustration and not by way of limitation.

### Example 1

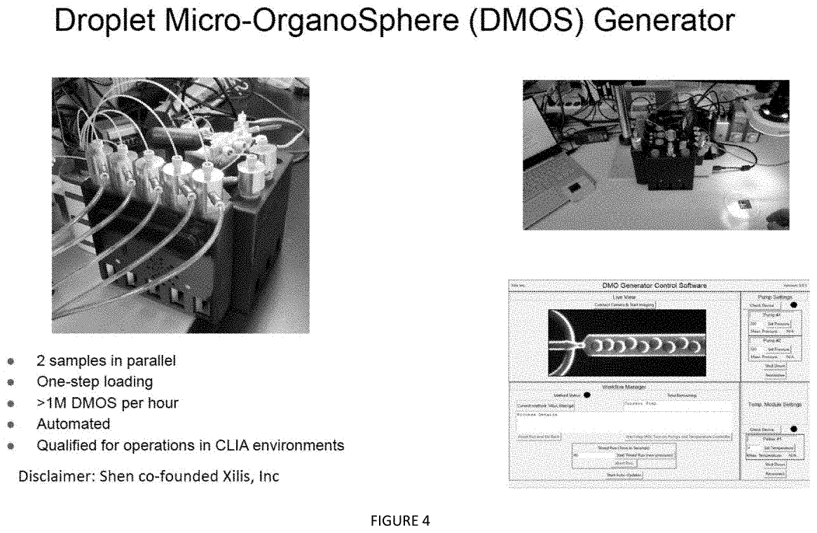

The present disclosure provides, in part, a technology termed (Droplet organoid-based Immuno-oncology Assay; DOIOA) that leverages droplet microfluidics for generating Patient-Derived Micro-Organospheres (see, FIG. 4). According to one embodiment, these PMOSs are generated and cultured to useability within one week and are co-cultured with matched immune-infiltrating T cells that are engineered a priori or isolated and expanded from tumor cells for testing immune checkpoint inhibitors (ICIs). It was observed that the assay is quite amenable to live cell imaging in real-time, with a clear established focal plane for identifying tumor cell killing by immune cells, and that immune cells can readily penetrate MATRIGEL droplets (see, FIG. 2). In fact, comparing a bulk organoid system to lung cancer PMOSs, it was surprisingly discovered that there was significantly more infiltration of MATRIGEL by PBMCs in the PMOS system over 72 hours than the bulk organoid system (see, FIG. 5).

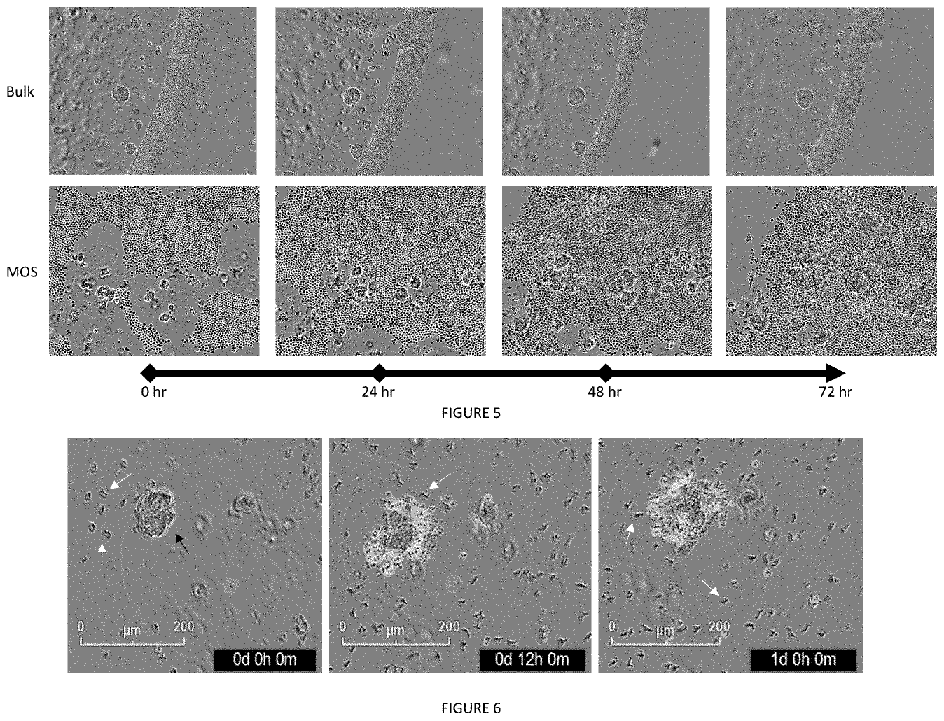

Apoptosis/cell death within PMOSs can be monitored in real time using intracellular dyes such as Annexin V Green (for apoptosis) and Cytotox Red (for cell death). For example, apoptosis and cell death can be seen in FIG. 6, wherein MHC-nonrestricted T acute lymphoblastic leukemia CD8+ T cells (TALL-104) are co-cultured with CRC organoids in the presence of intracellular dyes. In FIG. 6, white arrows are TALL-104 cells and the black arrow is a PMOS. Using intracellular dyes, PMBC has been shown to kill lung tumor PMOSs (data not shown). Alternative dyes include, but are not limited to, Caspase 3/7 and Cytotox for apoptosis and Cytolight Red for cell death.

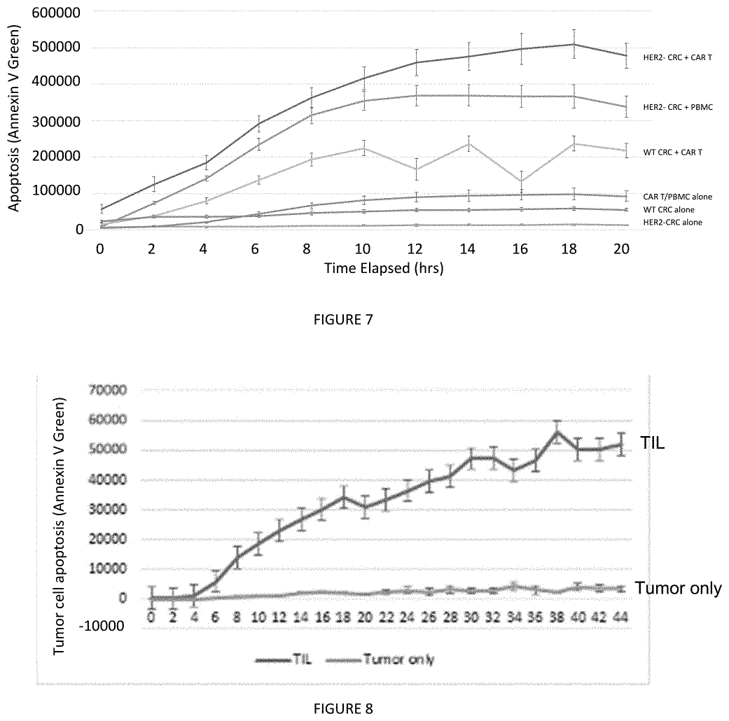

Using fluorescent dyes for apoptosis and cell death, DOIOA can reliably quantify tumor cell killing by immune cells (see, FIG. 7). Quantification of apoptotic (green) signal from co-culture confirms CAR-T-induced apoptosis of tumor cells. Higher rate and endpoint of apoptosis in experimental condition vs. HER2+ CRC with PBMCs and wildtype CRC with CAR Ts shows that this imaging assay is sensitive to those differences and can properly identify and quantify the differences between apoptosis and induced cell death.

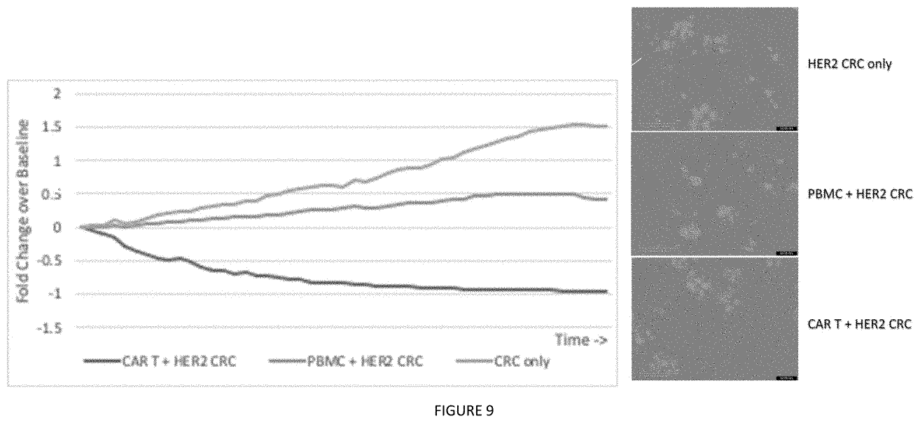

Given the small number of tumor cells required for the PMOS generation, this assay also minimizes the number of effector immune cells required to identify tumor cell killing. Using a CAR-T system against HER2-expressing CRC droplet organoids (expressing mCherry reporter), it was shown that DOIOA can identify and quantify CAR-T-specific killing of cognate HER2+ CRC cells (see, FIG. 9). Images taken with IncuCyte S3 over 48 hours shows clear difference between CAR-T-specific killing of HER2-expressing CRC droplet organoids and minimal killing by non-specific PBMC against HER2-expressing CRC droplet organoids. Increase in mCherry signal of HER2+ CRC in the absence of immune cells demonstrates the viability of CRC cells.

Additionally, one can culture match tumor infiltrating lymphocytes (TILs) and lung tumor organoids for a test of TIL potency (see, FIG. 8)

### Example 2

As described herein, a Micro-Organosphere assay for immune cytotoxicity (MOSAIC) can be used to quantify rapid-expansion phase TIL cytotoxicity against matched lung tumor PMOSs. By culturing tumor PMOSs with matched rapid-expansion phase TIL in the presence of Annexin V Green, immune-induced tumor cell apoptosis can be quantified and imaged.

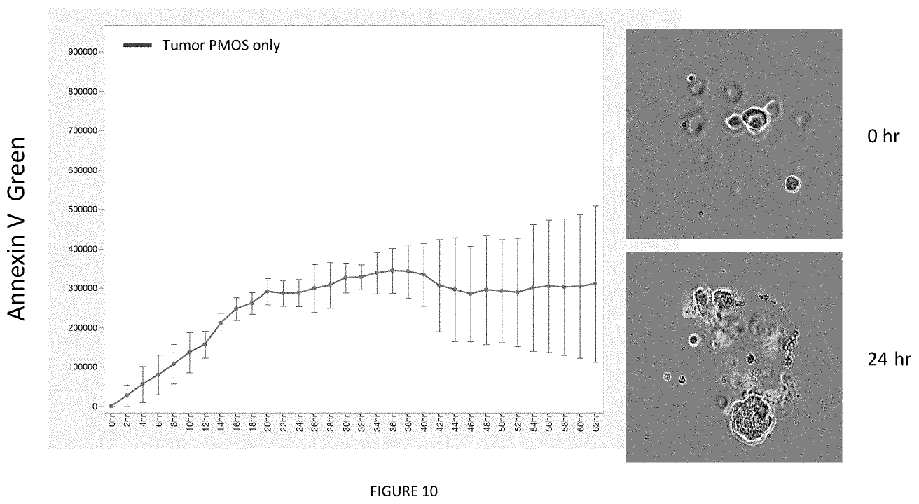

First, baseline apoptosis as a function of media conditions was assessed, as shown in FIG. 10. Once matched TIL were added, droplet infiltration and killing of tumor cells was observed, as seen in FIG. 11.

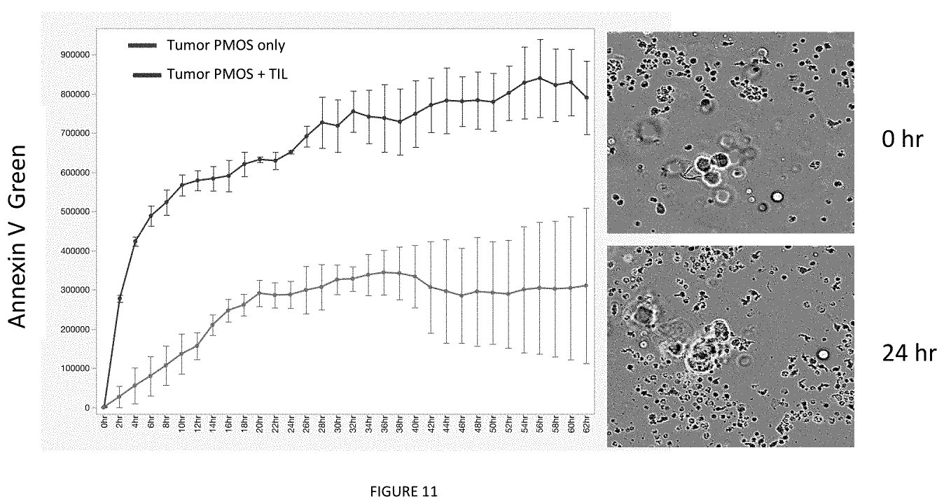

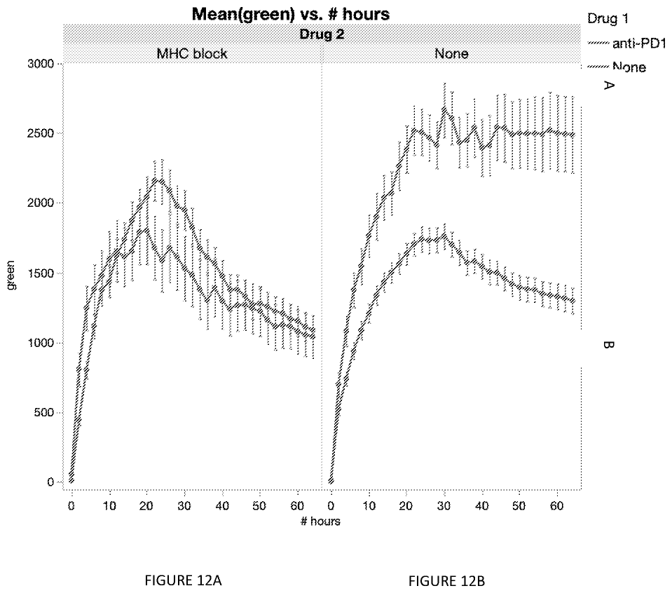

Advantageously, PMOSs can be used in a combination setting, wherein the interaction between tumor cells and T cells can be modulated. In this experiment, lung tumor PMOS was treated with the anti-PD1 drug Nivolumab with addition of matched TILs. The Immuno-Oncology assay data suggested anti-PD1 kills lung tumor PMOSs when TILs were added (FIG. 12B). In a parallel experiment, MHC I/II blocking antibodies were used to assess the antigen-specific killing enhanced by Nivolumab treatment, since the literature suggests that MHC-I/II plays an important role in spontaneous, PD-1 blockade-mediated antitumor immunity. When the PMOS is treated with MHC I/II blocking antibodies, the tumor killing effect observed previously disappeared (FIG. 12A).

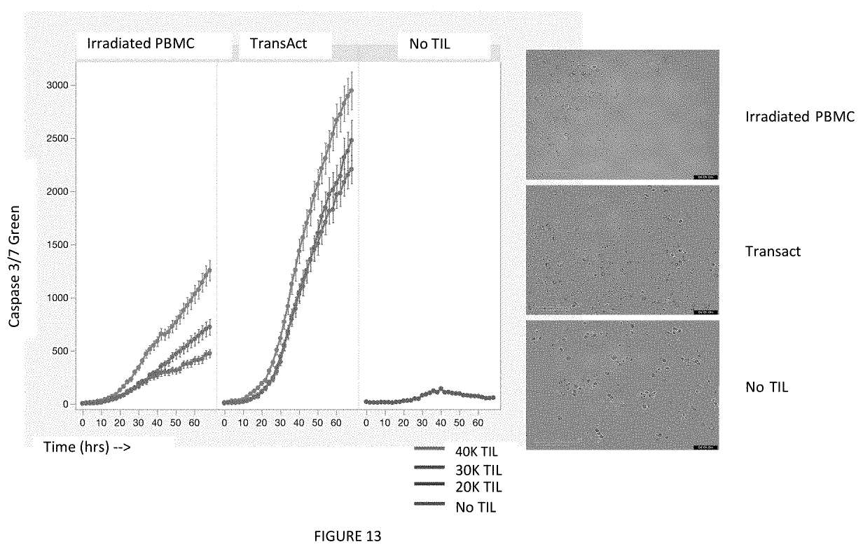

An interesting application of the MOSAIC assay is for evaluating changes to TIL manufacturing processes and selecting optimal protocols. In a collaboration with Scott Antonia and Cellular Biomedicine Group (CBMG), TIL subject to rapid expansion phase (REP) in the presence of irradiated PBMC feeder cells or TransAct T cell activator reagent was received. When co-cultured with tumor PMOS derived from the same patient (approximately 4000 PMOS per experiment), TIL expanded with TransAct are more cytotoxic against tumor PMOS than those expanded with the conventional method (irradiated PBMC) (see, FIG. 13). This could motivate better manufacturing processes of TIL and serve as a potency assay for validating those methods. Further, FIG. 13 supports the claim that cell death can be quantified in real-time. In each experiment, the amount of PMOSs were substantially the same and only the amount of TILs were varied. It is clear that there is statistically significant increased cell death with increasing amounts of TILs.

### Example 3

Clinical trials for anti-PD1-refractory metastatic NSCLC patients can make use of TIL adoptive T cell therapy (ACT) and explore the potency assay's predictive value as described below.

### TIL Selection and Tumor Cryopreservation

Tumor biopsies will be split into several pieces. One can be for TIL isolation and expansion (pre-REP; culture tumor single cell digest in RPMI-1640 or ImmunoCult Human T Cell Expansion Media in the presence of 3000-6000 U/mL IL-2) Another partition can be cryopreserved in FBS+10% DMSO and stored in LN2 until further use for the potency assay. Still another can be used for Micro-Organosphere establishment and culture, whereby the Micro-Organospheres are cryopreserved before use in the assay, or encapsulated directly in droplets when the TIL are ready to test.

### Thawing of Tumor Partition/Droplet Generation

Tumor cells can be thawed according to standard procedures for thawing mammalian cells and cultured in media specific to the cancer/organ type. After counting, single cells will be encapsulated into droplets and grown until sufficiently sized Micro-Organosphere are present in each droplet. At this point, the co-culture with TIL can be performed.

### Potency Assay (Co-Culture)

TIL that had been in culture or thawed and recovered in RPMI-1640+10% FBS+3000 U/mL IL-2 for greater than or equal to 2 days, will be seeded into a 96-well plate, wherein each well contains 40-50 droplets. The effector:target ratio will be varied in every assay to identify ratiometric differences in potency of TIL against matched tumor cells. The media can be 50% Micro-Organosphere media (depending on organ type) without any Y-27632 (used to promote Micro-Organosphere establishment but also an inhibitor of anoikis), and 50% TIL media. TIL media can depend on the manufacturer as many parties use different media. For example, PrimeXV T Cell Expansion XSFM from Fujifilm with 3-5% human platelet lysate or RPMI-1640+10% FBS can be used. The use of IL-2 in the co-culture is also context-dependent. If testing for the baseline potency of TIL, IL-2 is not added to the co-culture media. However, when testing CAR T or another engineered T cell for which antigen-specificity is known, IL-2 can be used in the co-culture media. Imaging can be performed using the IncuCyte S3 high-throughput fluorescent microscope to image a 96-well plate, taking 5 images per well every 1-2 hours over 2-3 days of co-culture. These co-cultures are in the presence of intracellular dyes as described herein.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. (canceled)

2. A method for determining the potency of tumor cell killing by effector immune cells, the method comprising:
forming Patient-Derived Micro-Organospheres (PMOSs) in a microfluidic device;
dispensing one or more of the PMOSs from the microfluidic device into a well of a plate;
co-culturing the one or more of the PMOSs and effector immune cells in a medium in the well; and
quantifying tumor cell killing of the one or more of the PMOSs by the effector immune cells in the medium.

3. The method of claim 2, the method further comprising isolating, freezing and storing the responding effector immune cells and/or tumor cells for further analysis in a high throughput and rapid manner.

4. The method of claim 2 in which the effector immune cells are selected from the group consisting of CAR-T cells, tumor infiltrating lymphocytes (TILs), Peripheral Blood Mononuclear Cells (PBMCs), T cells isolated from PBMCs, T cells isolated and expanded from tumor cells, and combinations thereof.

5. The method of claim 2, wherein the effector immune cells comprise TILs.

6. The method of claim 5, wherein the TILs are rapid-expansion phase (REP) TILs.

7. The method of claim 2, wherein the PMOSs are matched with the effector immune cells.

8. The method of claim 2, wherein the tumor cell killing by the effector immune cells is quantified in real time using fluorescent dyes.

9. The method of claim 8, wherein the fluorescent dyes comprise at least one of Annexin V Green, Caspase 3/7, Cytotox, Cytotox Red, Cytolight Red, orange color, or near-infrared color dyes.

10. The method of claim 2, further comprising measuring baseline apoptosis of the PMOSs as a function of the media conditions in the absence of the effector immune cells.

11. The method of claim 2, wherein the PMOSs are formed in the microfluidic device by a method selected from the group consisting of:
(I) combining a dissociated tissue sample and a fluid matrix material to form an unpolymerized mixture, forming a plurality of droplets of the unpolymerized mixture, and
polymerizing the droplets to form a plurality of PMOSs between 1 and 200 dissociated cells, distributed therein;
(II) combining a dissociated tissue sample and a fluid matrix material to form an unpolymerized mixture, forming a plurality of droplets having less than a 25% variation in a size of the droplets by converging a stream of the unpolymerized mixture with one or more streams of a fluid that is immiscible with the unpolymerized mixture, polymerizing the droplets to form a plurality of PMOSs between 1 and 200 dissociated cells distributed therein, and separating the plurality of PMOSs from the fluid that is immiscible;
(III) combining a dissociated tissue sample and a fluid matrix material to form an unpolymerized mixture, forming a plurality of droplets of the unpolymerized mixture having less than a 25% variation in a size of the droplets,
polymerizing the droplets to form a plurality of PMOSs with between 1 and 200 dissociated cells
cryopreserving the plurality of PMOSs;
(IV) combining a dissociated tissue sample and a fluid matrix material to form an unpolymerized mixture, forming a plurality of droplets of the unpolymerized mixture, polymerizing the droplets to form a plurality of PMOSs with between 1 and 200 dissociated cells distributed therein, and cryopreserving the plurality of PMOSs within 15 days; or
(V) combining a dissociated tissue sample and a fluid matrix material to form an unpolymerized mixture, forming a plurality of droplets having less than a 25% variation in a size of the droplets by converging a stream of the unpolymerized mixture with one or more streams of a fluid that is immiscible with the unpolymerized mixture, polymerizing the droplets to form a plurality of PMOSs with between 1 and 200 dissociated cells distributed therein, and
cryopreserving the PMOSs before six passages, whereby heterogeneity of the cells within the PMOSs is maintained.

12. The method of claim 11, wherein the dissociated tissue sample comprises one of: cells that are not stem cells; a biopsy sample from a metastatic tumor; a clinical tumor sample comprising both cancer cells and stroma cells; or tumor cells and one or more of mesenchymal cells, endothelial cells, and immune cells.

13. The method of claim 11, wherein combining the dissociated tissue sample and the fluid matrix material comprises combining the dissociated tissue sample with a basement membrane matrix.

14. The method of claim 11, wherein the dissociated tissue sample is combined with the fluid matrix material within six hours of removing the tissue sample from the patient.

15. (canceled)

16. The method of claim 2, wherein the PMOSs comprise dissociated tissue cells and a fluid matrix material, and the dissociated tissue cells are distributed within the fluid matrix material at a density of less than 1×10⁷ cells/ml.

17. The method of claim 16, wherein dissociated tissue cells are distributed within the fluid matrix material at a density of less than 5×10⁶ cells/ml.

18. The method of claim 16, wherein dissociated tissue cells are distributed within the fluid matrix material at a density of less than 1×10⁶ cells/ml.

19. The method of claim 16, wherein dissociated tissue cells are distributed within the fluid matrix material at a density of less than 5×10⁵ cells/ml.

20. The method of claim 2, wherein the dissociated tissue cells are dissociated tumor cells.

21. The method of claim 2, wherein the PMOSs are formed in the microfluidic device by:
combining a dissociated tissue sample and a fluid matrix material to form an unpolymerized mixture, forming a plurality of droplets from a continuous stream of the unpolymerized mixture wherein the droplets have less than a 25% variation in size, and
polymerizing the droplets by warming to form the plurality of PMOSs.

22. The method of claim 2, wherein the PMOSs are formed in the microfluidic device by a method selected from the group consisting of:
(I) combining a dissociated tissue sample and the fluid matrix material to form an unpolymerized mixture, forming a plurality of droplets of the unpolymerized mixture, and
polymerizing the droplets to form a plurality of PMOSs each having a diameter of between 50 μm and 350 μm;
(II) combining a dissociated tissue sample and the fluid matrix material to form an unpolymerized mixture, forming a plurality of droplets having less than a 25% variation in a size of the droplets by converging a stream of the unpolymerized mixture with one or more streams of a fluid that is immiscible with the unpolymerized mixture, polymerizing the droplets to form a plurality of PMOSs each having a diameter of between 50 μm and 350 μm; and
separating the plurality of PMOSs from the fluid that is immiscible;
(III) combining a dissociated tissue sample and the fluid matrix material to form an unpolymerized mixture, forming a plurality of droplets of the unpolymerized mixture having less than a 25% variation in a size of the droplets,
polymerizing the droplets to form a plurality of PMOSs each having a diameter of between 50 μm and 350 μm; and
cryopreserving the plurality of PMOSs;
(IV) combining a dissociated tissue sample and the fluid matrix material to form an unpolymerized mixture, forming a plurality of droplets of the unpolymerized mixture, polymerizing the droplets to form a plurality of PMOSs each having a diameter of between 50 μm and 350 μm; and
cryopreserving the plurality of PMOSs within 15 days; or
(V) combining a dissociated tissue sample and the fluid matrix material to form an unpolymerized mixture, forming a plurality of droplets having less than a 25% variation in a size of the droplets by converging a stream of the unpolymerized mixture with one or more streams of a fluid that is immiscible with the unpolymerized mixture, polymerizing the droplets to form a plurality of PMOSs each having a diameter of between 50 μm and 350 μm; and
cryopreserving the PMOSs before six passages, whereby heterogeneity of the cells within the PMOSs is maintained.

23. The method of claim 22, wherein the plurality of PMOSs each have a diameter of between 50 μm and 250 μm.

24. The method of claim 23, wherein the plurality of PMOSs each have a diameter of between 50 μm and 200 μm.

25. The method of claim 22, wherein the dissociated tissue sample comprises one of: cells that are not stem cells; a biopsy sample from a metastatic tumor; a clinical tumor sample comprising both cancer cells and stroma cells; or tumor cells and one or more of mesenchymal cells, endothelial cells, and immune cells.

26. The method of claim 22, wherein combining the dissociated tissue sample and the fluid matrix material comprises combining the dissociated tissue sample with a basement membrane matrix.

27. The method of claim 22, wherein the dissociated tissue sample is combined with the fluid matrix material within six hours of removing the tissue sample from the patient.
